(19) United States
(12) Patent Application Publication
(10) Pub. No.: US 2024/0207609 A1
(43) Pub. Date: Jun. 27, 2024

(54) ELECTRICAL STIMULATION DEVICE

(71) Applicant: GIMER MEDICAL CO., LTD., New Taipei City (TW)

(72) Inventors: Ya-Shin Ko, New Taipei City (TW); Chi-Yu Huang, New Taipei City (TW); Wei-Tso Lin, New Taipei City (TW)

(21) Appl. No.: 18/390,444

(22) Filed: Dec. 20, 2023

Related U.S. Application Data
(60) Provisional application No. 63/435,333, filed on Dec. 27, 2022.

(51) Int. Cl. A61N 1/36; A61N 1/02

(52) U.S. Cl. CPC A61N 1/36; A61N 1/025

(57) ABSTRACT

An electrical stimulation device includes a control circuit, a power supply circuit, and an electrical stimulation circuit. The power supply circuit provides a first power supply signal. The electrical stimulation circuit is controlled by the control circuit to generate an electrical stimulation signal according to the first power supply signal. The electrical stimulation signal includes two burst signals. A burst duty cycle of the first burst signal ranges from 0.0005% to 50%. A burst frequency of the first burst signal ranges from 0.1 Hz to 200 Hz. The burst signals are monophasic burst signals with opposite polarities or both are biphasic burst signals. Pulse frequencies of pulses in the monophasic burst signals and the biphasic burst signals range from 1000 Hz to 10 million Hz. Pulse duty cycles of the pulses in the monophasic burst signals and the biphasic burst signals range from 0.01% to 50%.

### CROSS-REFERENCE TO RELATED APPLICATION

This application claims the benefit of U.S. provisional application Ser. No. 63/435,333, filed on Dec. 27, 2022. The entirety of the above-mentioned patent application is hereby incorporated by reference herein and made a part of the specification.

### BACKGROUND

### Technical Field

The disclosure relates to an electrical stimulation technology, and particularly, to an electrical stimulation device.

### Related Art

In recent years, a plurality of electrical stimulation devices are developed. Because of development of precise manufacturing technologies, medical instruments (for example, an electrical stimulation device) are miniaturized in size. In order to meet requirements of miniaturization, a power supply circuit (such as battery) inside the electrical stimulation device also needs to be reduced in size, which will affect the power supply capability, such as the power that the power supply circuit can provide will be relatively low. Usually, an electrical stimulation signal of the electrical stimulation device has a low frequency, such as the frequency of the electrical stimulation signal is not greater than a range from 200 to 300 Hz. Since the low power consumption requirements for the low frequency, the power supply circuit with a lower power can meet the electric power required by the electrical stimulation device that generating the low frequency electrical stimulation. However, when a high-frequency electrical stimulation (such as not less than 1000 Hz) is required, a low-power power supply circuit may not be able to meet the requirements of an electrical stimulation device that generates the high-frequency electrical stimulation signal due to the high power consumption requirements. Therefore, how to improve an electrical stimulation device to enable a power supply circuit with a lower power to meet electric power required for the electrical stimulation device for generating the high-frequency electrical stimulation signal is an important topic.

### SUMMARY

In view of the foregoing, the disclosure provides an electrical stimulation device. In some embodiments, an electrical stimulation device includes a control circuit, a power supply circuit, and an electrical stimulation circuit. The power supply circuit provides a first power supply signal. The electrical stimulation circuit is controlled by the control circuit to generate an electrical stimulation signal according to the first power supply signal. The electrical stimulation signal includes a first burst signal and a second burst signal. A burst duty cycle of the first burst signal ranges from 0.0005% to 50%, and a burst frequency of the first burst signal ranges from 0.1 Hz to 200 Hz. The first burst signal is a first monophasic burst signal, and the first monophasic burst signal includes a plurality of first pulses. The second burst signal is a second monophasic burst signal, and the second monophasic burst signal includes a plurality of second pulses. The first pulses have a polarity opposite to that of the second pulses. The first pulses have a first pulse frequency, and the second pulses have a second pulse frequency. Both the first pulse frequency and the second pulse frequency range from 1000 Hz to 10 million Hz. The first pulses have a pulse duty cycle ranging from 0.01% to 50%.

In some embodiments, an electrical stimulation device includes a control circuit, a power supply circuit, and an electrical stimulation circuit. The power supply circuit provides a first power supply signal. The electrical stimulation circuit is controlled by the control circuit to generate an electrical stimulation signal according to the first power supply signal. The electrical stimulation signal includes a first burst signal and a second burst signal. A burst duty cycle of the first burst signal ranges from 0.0005% to 50%, and a burst frequency of the first burst signal ranges from 0.1 Hz to 200 Hz. Each of the first burst signal and the second burst signal is a biphasic burst signal, and the biphasic burst signal includes a plurality of first pulses and a plurality of second pulses occurring alternately. The first pulses have a polarity opposite to that of the second pulses. The first pulses have a first pulse frequency, and the second pulses have a second pulse frequency. Both the first pulse frequency and the second pulse frequency range from 1000 Hz to 10 million Hz. The first pulses have a duty cycle ranging from 0.01% to 50%.

In summary, according to some embodiments, the power supply circuit with a low power (such as the power ranges from 0.1 milliwatts (mW) to 30 milliwatts or ranges from 0.1 milliwatts to 30 watts) of the disclosure can meet electric power required for the electrical stimulation device for generating an high-frequency electrical stimulation signal (such as the pulse has a frequency ranging from 1000 Hz to 10 million Hz). In some embodiments, by adjusting the burst duty cycle of the first burst signal and/or the second burst signal to a range from 0.0005% to 50%, adjusting the burst frequency of the first burst signal and/or the second burst signal to a range from 0.1 Hz to 200 Hz, and adjusting the pulse duty cycle of the first pulse and/or the second pulse to a range from 0.01% to 50%, power consumption of the electrical stimulation device can be reduced and power can be saved. In some embodiments, when the first burst signal and the second burst signal are monophasic burst signals with opposite polarities, the first burst signal and the second burst signal that occur alternately can implement a biphasic charge balance, and the first burst signal and the second burst signal that occur alternately and are spaced apart by a period of time can implement a biphasic charge balance with delay. In this embodiment, damage caused by the electrical stimulation signal to a tissue of an organism can be avoided as much as possible. In some embodiments, when the first burst signal and the second burst signal are biphasic burst signals, the first pulses and the second pulses occurring alternately in each of the first burst signal and the second burst signal can implement a biphasic charge balance, and the first burst signal and the second burst signal that occur alternately and are spaced apart by a period of time can implement a biphasic charge balance with delay. In this way, damage caused by the electrical stimulation signal to a tissue of an organism can be further avoided as much as possible.

### BRIEF DESCRIPTION OF THE DRAWINGS

FIG. 1A is a schematic diagram of an electrical stimulation device according to some embodiments of the disclosure.

FIG. 1B is a schematic diagram of an electrical stimulation device being a transcutaneous electrical stimulation device according to some embodiments of the disclosure.

FIG. 1C is a schematic diagram of an electrical stimulation device being a minimum-invasive electrical stimulation device according to some embodiments of the disclosure.

FIG. 1D is a schematic diagram of an electrical stimulation device being an implantable electrical stimulation device according to some embodiments of the disclosure.

FIG. 2 is a schematic diagram of an electrical stimulation signal according to a first embodiment of the disclosure.

FIG. 3A is a schematic diagram of an electrical stimulation signal according to a second embodiment of the disclosure.

FIG. 3B is a schematic diagram of an electrical stimulation signal according to a third embodiment of the disclosure.

FIG. 4 is a schematic diagram of an electrical stimulation device according to some embodiments of the disclosure.

FIG. 5 is a detailed schematic circuit diagram of an electrical stimulation circuit according to some embodiments of the disclosure.

FIG. 6 is a schematic diagram of an electrode detection circuit and part of an electrical stimulation circuit according to some embodiments of the disclosure.

FIG. 7 is a schematic diagram of a discharging circuit and part of an electrical stimulation circuit according to some embodiments of the disclosure.

FIG. 8 is a schematic diagram of physiological symptom improvements of users after using an electrical stimulation device implemented by a transcutaneous electrical stimulation device according to some embodiments of the disclosure.

### DETAILED DESCRIPTION

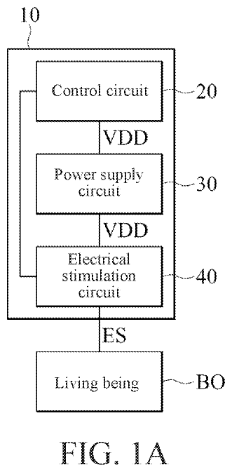

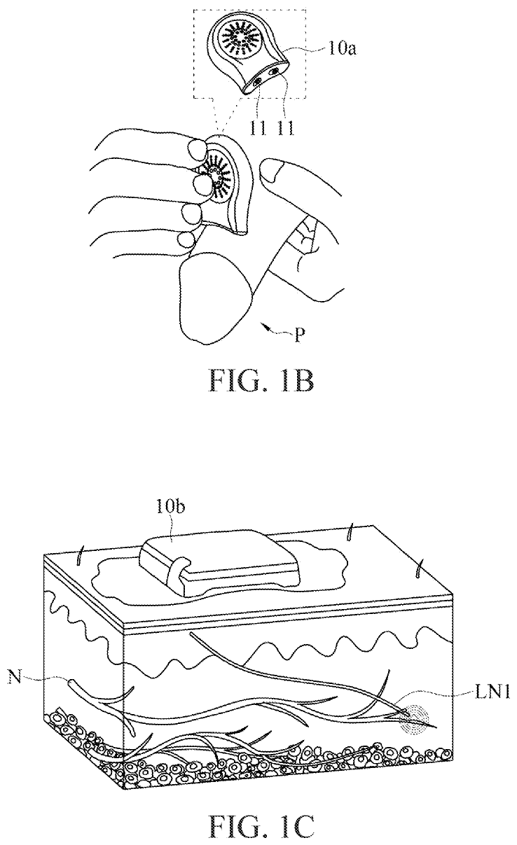

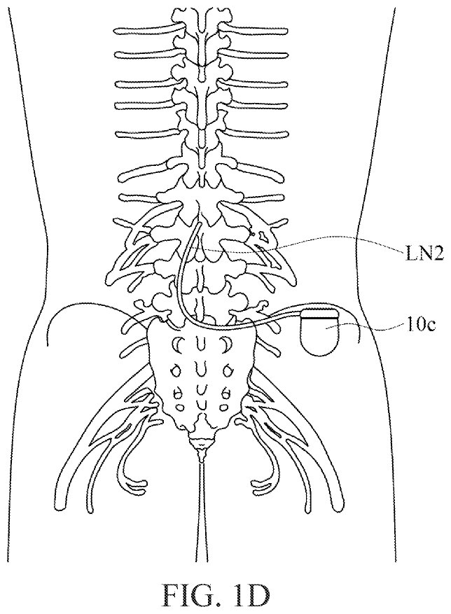

FIG. 1A is a schematic diagram of an electrical stimulation device 10 according to some embodiments of the disclosure. The electrical stimulation device 10 includes a control circuit 20, a power supply circuit 30, and an electrical stimulation circuit 40. The power supply circuit 30 is coupled to the control circuit 20 and the electrical stimulation circuit 40. The control circuit 20 is coupled to the electrical stimulation circuit 40. The power supply circuit 30 provides a first power supply signal VDD, and transmits the first power supply signal VDD to the control circuit 20 and the electrical stimulation circuit 40, to supply power to the control circuit 20 and the electrical stimulation circuit 40. The electrical stimulation circuit 40 is controlled by the control circuit 20 to generate an electrical stimulation signal ES according to the first power supply signal VDD to perform electrical stimulation on an organism BO (such as a certain nerve thereof).

The electrical stimulation device 10 may be an electrical stimulation device in a type such as a transcutaneous type, a minimum-invasive type, or an implantable type. Situations in which the electrical stimulation device 10 is an electrical stimulation device in a type such as a transcutaneous type, a minimum-invasive type, or an implantable type are described below with reference to FIG. 1B to FIG. 1D respectively.

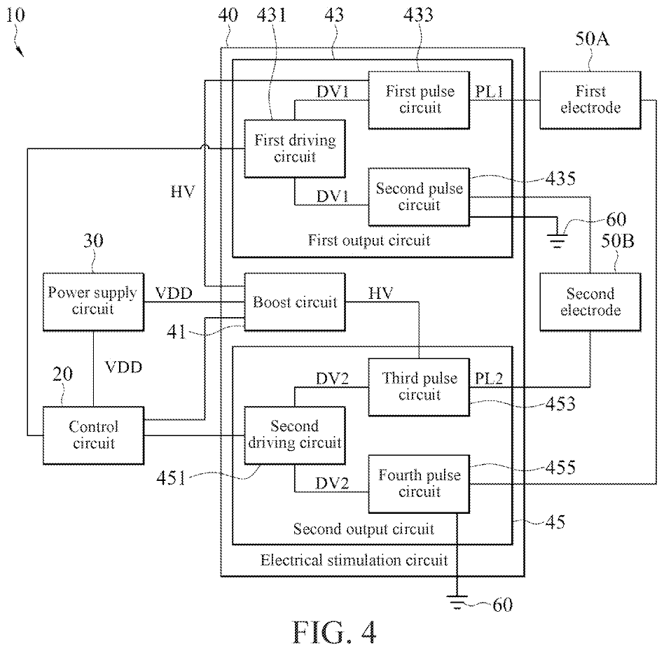

FIG. 1B is a schematic diagram of an electrical stimulation device 10 being a transcutaneous electrical stimulation device 10a according to some embodiments of the disclosure. When the electrical stimulation device 10 is a transcutaneous electrical stimulation device, an electrical stimulation signal ES can stimulate superficial nerves of tissues of an organism through an electrode or an electrode pad, such as superficial nerves that are in an upper limb, a lower limb, and a trunk and whose depths are about 1, 2, or 3 centimeters under the skin. As shown in FIG. 1B, in an example, the electrical stimulation device 10 implemented by the transcutaneous electrical stimulation device 10a has a pair of electrodes (namely, electrode pair 11) (which can implement a first electrode 50A and a second electrode 50B shown in FIG. 4 to FIG. 6), to output a biphasic electrical stimulation signal (which implements the electrical stimulation signal ES of the electrical stimulation device 10 shown in FIG. 1A) to perform bipolar electrical stimulation or to output a monophasic electrical stimulation signal (which implements the electrical stimulation signal ES of the electrical stimulation device 10 shown in FIG. 1A) to perform monopolar electrical stimulation. If a superficial nerve close to a reproductive organ P of a male is used as an example, the superficial nerve may be a dorsal penile nerve between a corona of glans penis and the middle of the reproductive organ P. The superficial nerve may alternatively be a perineal nerve, a pudendal nerve, an ilioinguinal nerve, a vagina, or the like. In this embodiment, some physiological symptoms of the organisms can be alleviated such as nerve overactive symptoms, premature ejaculation, vulvar vestibulitis, overactive bladder, or urinary incontinence of a human body.

FIG. 1C is a schematic diagram of an electrical stimulation device 10 being a minimum-invasive electrical stimulation device 10b according to some embodiments of the disclosure. In an example, the electrical stimulation device 10 implemented by the minimum-invasive electrical stimulation device 10b (such as an electrical stimulator for a peripheral nerve) is affixed to the skin, to emit energy (such as the electrical stimulation signal ES of the electrical stimulation device 10 shown in FIG. 1A) in a wireless or wired manner and transmit the energy to a minimum-invasive electrode lead LN1 implanted under the skin, to electrically stimulate a nerve (namely, target nerve N) close to the end of the electrode lead LN1 to relieve pains or promote nerve repair and muscle repair, to improve the control from the human body over muscles and recover or strengthen the muscle force. One end of the electrode lead LN1 can be implanted near the target nerve N, and the distal end of the electrode LN1 can be placed subcutaneously and near the minimally invasive electrical stimulation device 10b above the skin to transmit energy wirelessly (as shown in FIG. 1C) or the distal end of the electrode lead LN1 can be stretched out from the surface of the skin and connected to the minimum-invasive electrical stimulation device 10b by wired transmission.

FIG. 1D is a schematic diagram of an electrical stimulation device 10 being an implantable electrical stimulation device 10c according to some embodiments of the disclosure. The electrical stimulation device 10 implemented by the implantable electrical stimulation device 10c (such as an electrical stimulator for spinal marrow) may perform electrical stimulation on spinal marrow by implanting an electrode lead LN2 into an epidural space of a spine and using an electrical stimulation signal ES (as shown in FIG. 1A), to achieve a nerve regulation objective.

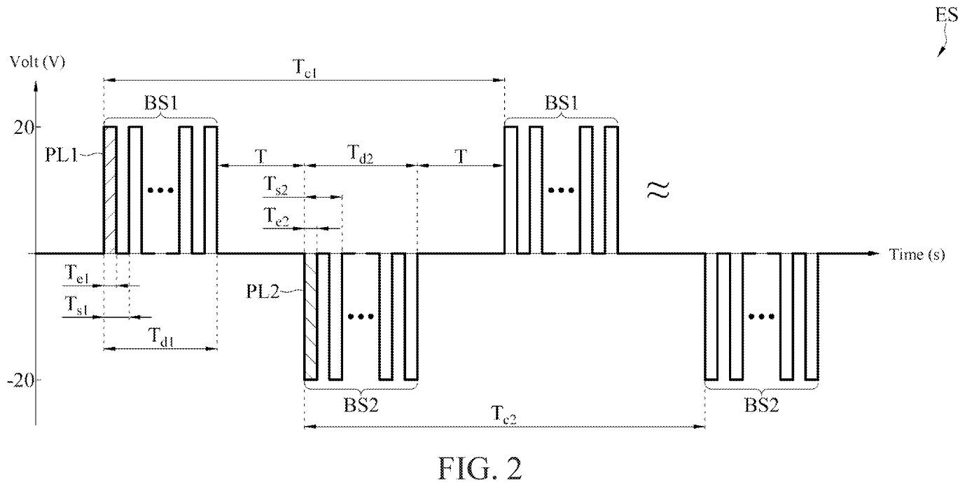

FIG. 2 is a schematic diagram of an electrical stimulation signal ES according to a first embodiment of the disclosure. The electrical stimulation signal ES includes a first burst signal BS1 and a second burst signal BS2. The first burst signal BS1 has a burst duty cycle (hereinafter referred to as a first burst duty cycle) ranging from 0.0005% to 50%. As shown in Formula 1, a burst duty cycle (T_{bdc}) of a certain burst signal refers to a burst width (T_{d}) of the certain burst signal divided by a burst cycle (T_{c}) of the certain burst signal. For example, the first burst duty cycle of the first burst signal BS1 refers to a burst width (hereinafter referred to as a first burst width T_{d1}) of the first burst signal BS1 divided by a burst cycle (hereinafter referred to as a first burst cycle T_{c1}) of the first burst signal BS1. The first burst signal BS1 has a burst frequency (hereinafter referred to as a first burst frequency) ranging from 0.1 Hz to 200 Hz. As shown in Formula 2, a burst frequency (T_{bf}) of a certain burst signal refers to a reciprocal of a burst cycle (T_{c}) of the certain burst signal. For example, the first burst frequency of the first burst signal BS1 refers to a reciprocal of the first burst cycle T_{c1} of the first burst signal BS1. In an example, the first burst duty cycle is 1%, the first burst width T_{d1} is 10 milliseconds (ms), the first burst cycle T_{c1} is 1 second, and the first burst frequency is 1 Hz.

$$\frac{{T}_{d}}{{T}_{c}} = {T}_{bdc} \qquad ( Formula 1 )$$

$$\frac{1}{{T}_{c}} = {T}_{bf} \qquad ( Formula 2 )$$

The first burst signal BS1 is a first monophasic burst signal. The first monophasic burst signal includes a plurality of first pulses PL1 (for clear description, one of the plurality of first pulses PL1 is represented with an oblique line from lower left to upper right in FIG. 2). The second burst signal BS2 is a second monophasic burst signal. The second monophasic burst signal includes a plurality of second pulses PL2 (for clear description, one of the plurality of second pulses PL2 is represented with an oblique line from upper left to lower right in FIG. 2). The first pulses PL1 have a polarity opposite to that of the second pulses PL2. That is to say, the first monophasic burst signal has a polarity opposite to that of the second monophasic burst signal. For example, polarities of both the first monophasic burst signal and the first pulses PL1 are positive, and polarities of both the second monophasic burst signal and the second pulses PL2 are negative, but the disclosure is not limited thereto. The polarities of the first monophasic burst signal and the first pulses PL1 may be negative, and the polarities of the second monophasic burst signal and the second pulses PL2 may be positive.

The first pulses PL1 have a first pulse frequency. The second pulses PL2 have a second pulse frequency. As shown in Formula 3, a pulse frequency (T_{pf}) of a certain pulse refers to a reciprocal of a pulse cycle (Tₛ) of the certain pulse. For example, the first pulse frequency of the first pulse PL1 refers to a reciprocal of a pulse cycle (hereinafter referred to as a first pulse cycle Tₛ₁) of the first pulse PL1, and the second pulse frequency of the second pulse PL2 refers to a reciprocal of a pulse cycle (hereinafter referred to as a second pulse cycle Tₛ₂) of the second pulse PL2. Both the first pulse frequency and the second pulse frequency range from 1000 Hz to 10 million Hz, namely, are in a range of high frequencies. The first pulses PL1 have a pulse duty cycle (hereinafter referred to as a first pulse duty cycle) ranging from 0.01% to 50%. As shown in Formula 4, a pulse duty cycle (T_{pdc}) of a certain pulse refers to a pulse width (Tₑ) of the certain pulse divided by a pulse cycle (Tₛ) of the certain pulse. For example, the first pulse duty cycle of the first pulse PL1 refers to a pulse width (hereinafter referred to as a first pulse width Tₑ₁) of the first pulse PL1 divided by the first pulse cycle Tₛ₁ of the first pulse PL1. In an example, both the first pulse frequency and the second pulse frequency are 500 thousand Hz, both the first pulse cycle Tₛ₁ and the second pulse cycle Tₛ₂ are 2 microseconds, the first pulse duty cycle is 9%, and the first pulse width Tₑ₁ is 0.18 microseconds.

$$\frac{1}{{T}_{s}} = {T}_{pf} \qquad ( Formula 3 )$$

$$\frac{{T}_{e}}{{T}_{s}} = {T}_{pdc} \qquad ( Formula 4 )$$

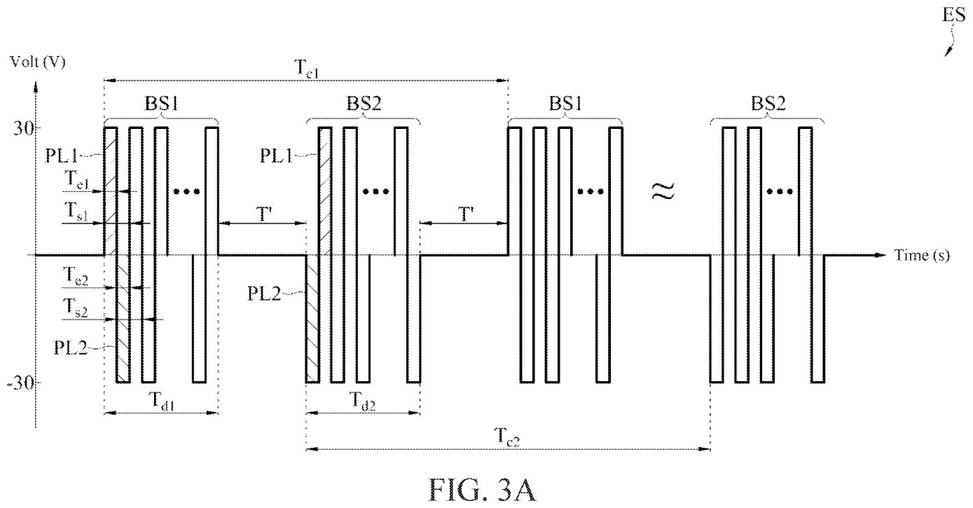

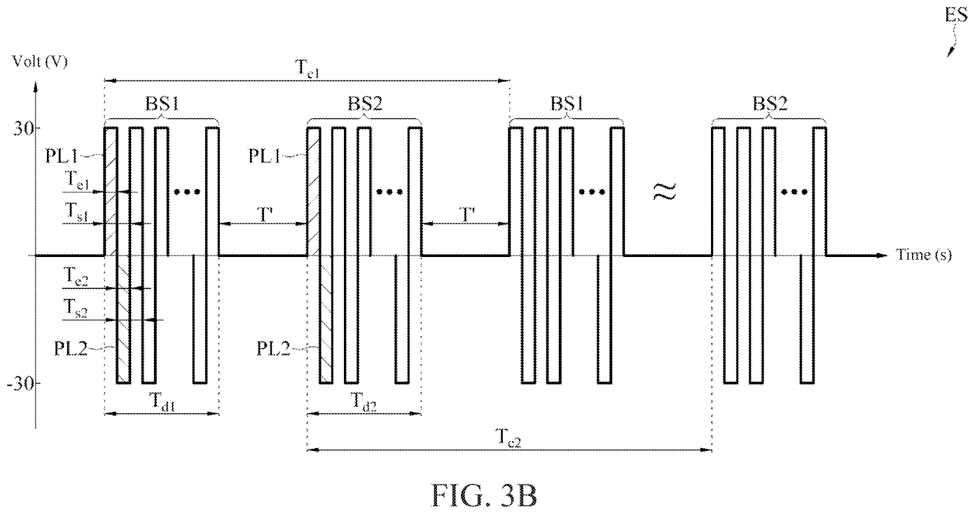

Referring to FIG. 3A and FIG. 3B, FIG. 3A is a schematic diagram of an electrical stimulation signal ES according to a second embodiment of the disclosure, and FIG. 3B is a schematic diagram of an electrical stimulation signal ES according to a third embodiment of the disclosure. Different from the first embodiment, in the second embodiment and the third embodiment, each of a first burst signal BS1 and a second burst signal BS2 is a biphasic burst signal. The biphasic burst signal includes a plurality of first pulses PL1 (for clear description, one of the plurality of first pulses PL1 is represented with an oblique line from lower left to upper right in FIG. 3A and FIG. 3B) and a plurality of second pulses PL2 (for clear description, one of the plurality of second pulses PL2 is represented with an oblique line from upper left to lower right in FIG. 3A and FIG. 3B) whose positive and negative phases occur alternately.

In this way, the electrical stimulation device 10 of the disclosure can generate a high-frequency signal (for example, the first pulse PL1 having the first pulse frequency and the second pulse PL2 having the second pulse frequency) to stimulate a tissue of an organism, and only electric power with a low power (such as the electric power provided by the first power supply signal VDD of the power supply circuit 30) can meet electric power required for generating the high-frequency signal. Furthermore, by adjusting the first burst duty cycle of the first burst signal BS1 to a range from 0.0005% to 50%, adjusting the first burst frequency of the first burst signal BS1 to a range from 0.1 Hz to 200 Hz, and adjusting the first pulse duty cycle of the first pulses PL1 to a range from 0.01% to 50%, power consumption of the electrical stimulation device 10 can be reduced and power can be saved.

As shown in FIG. 2, FIG. 3A, and FIG. 3B, in some embodiments, similar to the first burst signal BS1, the second burst signal BS2 has a burst duty cycle (hereinafter referred to as a second burst duty cycle) ranging from 0.0005% to 50%. The second burst duty cycle of the second burst signal BS2 refers to a burst width (hereinafter referred to as a second burst width T_{d2}) of the second burst signal BS2 divided by a burst cycle (hereinafter referred to as a second burst cycle T_{c2}) of the second burst signal BS2. The second burst signal BS2 has a burst frequency (hereinafter referred to as a second burst frequency) ranging from 0.1 Hz to 200 Hz. The second burst frequency of the second burst signal BS2 refers to a reciprocal of the second burst cycle T_{c2} of the second burst signal BS2. In an example, the second burst duty cycle is 1%, the second burst width T_{d2} is 10 milliseconds, the second burst cycle T_{c2} is 1 second, and the second burst frequency is 1 Hz. In this embodiment, power consumption of the electrical stimulation device 10 can be reduced and power can be saved. In some embodiments, the first burst duty cycle and the second burst duty cycle may be a same value or different values. In some embodiments, the first burst frequency and the second burst frequency may be a same value or different values.

As shown in FIG. 2, FIG. 3A, and FIG. 3B, in some embodiments, similar to the first pulse PL1, the second pulses PL2 have a pulse duty cycle (hereinafter referred to as a second pulse duty cycle) ranging from 0.01% to 50%. The second pulse duty cycle of the second pulse PL2 refers to a pulse width (hereinafter referred to as a second pulse width Tₑ₂) of the second pulse PL2 divided by the second pulse cycle Tₛ₂ of the second pulse PL2. In an example, the second pulse frequency is 500 thousand Hz, the second pulse cycle Tₛ₂ is 2 microseconds, the second pulse duty cycle is 9%, and the second pulse width Tₑ₂ is 0.18 microseconds. In this embodiment, a power consumption of the electrical stimulation device 10 can be reduced and power can be saved. In some embodiments, the first pulse duty cycle and the second pulse duty cycle may be a same value or different values. In some embodiments, the first pulse frequency and the second pulse frequency may be a same value or different values.

As shown in Formula 5, in some embodiments, a burst width (T_{d}) of a certain burst signal is a reciprocal of a burst frequency (T_{bf}) of the certain burst signal (namely, a burst cycle T_{c} of the certain burst signal) multiplied by a burst duty cycle (T_{bdc}) of the certain burst signal. As shown in FIG. 2, FIG. 3A, and FIG. 3B, in some embodiments, the first burst width T_{d1} of the first burst signal BS1 ranges from 25×10⁻⁹ seconds to 5 seconds. The 25×10⁻⁹ seconds are obtained by multiplying a reciprocal of a maximum first burst frequency by a minimum first burst duty cycle, the maximum first burst frequency is 200 Hz, and the minimum first burst duty cycle is 0.0005%. The 5 seconds are obtained by multiplying a reciprocal of a minimum first burst frequency by a maximum first burst duty cycle, the minimum first burst frequency is 0.1 Hz, and the maximum first burst duty cycle is 50%. In some embodiments, similar to the first burst width T_{d1}, the second burst width T_{d2} ranges from 25×10⁻⁹ seconds to 5 seconds. The 25×10⁻⁹ seconds are obtained by multiplying a reciprocal of a maximum second burst frequency by a minimum second burst duty cycle, the maximum second burst frequency is 200 Hz, and the minimum second burst duty cycle is 0.0005%. The 5 seconds are obtained by multiplying a reciprocal of a minimum second burst frequency by a maximum second burst duty cycle, the minimum second burst frequency is 0.1 Hz, and the maximum second burst duty cycle is 50%. In this embodiment, a power consumption of the electrical stimulation device 10 can be reduced and power can be saved. In some embodiments, the first burst width T_{d1} and the second burst width T_{d2} may be a same value or different values.

$$\frac{1}{{T}_{bf}} \times {T}_{bdc} = {T}_{c} \times {T}_{bdc} = {T}_{d} \qquad ( Formula 5 )$$

As shown in FIG. 2, FIG. 3A, and FIG. 3B, in some embodiments, the first pulse width Tₑ₁ of each first pulse PL1 ranges from 25×10⁻¹⁶ seconds to 5×10⁻³ seconds. The 25×10⁻¹⁶ seconds are obtained by dividing a minimum burst width (for example, a minimum first burst width T_{d1} or a minimum second burst width T_{d2}) by a maximum first pulse frequency (as shown in Formula 6), and the 5×10⁻³ seconds are obtained by dividing a maximum burst width (for example, a maximum first burst width T_{d1} or a maximum second burst width T_{d2}) by a minimum first pulse frequency (as shown in Formula 7). In Formula 6, T_{dmin} is the minimum burst width and is 25×10⁻⁹ seconds, and T_{pf1max} is the maximum first pulse frequency and is 10 million Hz. In Formula 7, T_{dmax} is the maximum burst width and is 5 seconds, and T_{pf1min} is the minimum first pulse frequency and is 1000 Hz. In some embodiments, similar to the first pulse width Tₑ₁, the second pulse width Tₑ₂ of each second pulse PL2 ranges from 25×10⁻¹⁶ seconds to 5×10⁻³ seconds. The 25×10⁻¹⁶ seconds are obtained by dividing a minimum burst width (for example, a minimum first burst width T_{d1} or a minimum second burst width T_{d2}) by a maximum second pulse frequency (as shown in Formula 8), and the 5×10−3 seconds are obtained by dividing a maximum burst width (for example, a maximum first burst width T_{d1} or a maximum second burst width T_{d2}) by a minimum second pulse frequency (as shown in Formula 9). In Formula 8, T_{dmin} is the minimum burst width and is 25×10⁻⁹ seconds, and T_{pf2max} is the maximum second pulse frequency and is 10 million Hz. In Formula 9, T_{dmax} is the maximum burst width and is 5 seconds, and T_{pf2min} is the minimum second pulse frequency and is 1000 Hz. In this embodiment, power consumption of the electrical stimulation device 10 can be reduced and power can be saved. In some embodiments, the first pulse width Tₑ₁ and the second pulse width Tₑ₂ may be a same value or different values.

$$\frac{{T}_{dmin}}{{T}_{pf 1 max}} = 25 \times 1 {0}^{- 16} s \qquad ( Formula 6 )$$

$$\frac{{T}_{dmax}}{{T}_{pf 1 min}} = 5 \times 1 {0}^{- 3} s \qquad ( Formula 7 )$$

$$\frac{{T}_{dmin}}{{T}_{pf 2 max}} = 25 \times 1 {0}^{- 16} s \qquad ( Formula 8 )$$

$$\frac{{T}_{dmax}}{{T}_{pf 2 min}} = 5 \times 1 {0}^{- 3} s \qquad ( Formula 9 )$$

In some embodiments, the control circuit 20 can be implemented by a high-performance operational circuit, such as a central processing unit. However, the disclosure is not limited thereto. To save power, the control circuit 20 can be implemented by a low-performance (therefore, low-cost) operational circuit, such as a microcontroller. In some embodiments, the control circuit 20 can control the electrical stimulation circuit 40 to change various signal parameters (for example, a burst duty cycle, a burst frequency, a pulse duty cycle, a pulse frequency, and the like) of the electrical stimulation signal ES. For example, the control circuit 20 can store, in a memory thereof, various setting parameters corresponding to various signal parameters or receive various setting parameters corresponding to various signal parameters that are input after a user operates a user interface (not shown in the figure), and the control circuit 20 changes the corresponding signal parameters of the electrical stimulation signal ES according to the setting parameters.

In some embodiments, when each of the first burst signal BS1 and the second burst signal BS2 is a biphasic burst signal, a starting pulse of the first burst signal BS1 may have a polarity opposite to or the same as that of a starting pulse of the second burst signal BS2. For example, as shown in FIG. 3A, a starting pulse of the first burst signal BS1 has a polarity opposite to that of a starting pulse of the second burst signal BS2. Specifically, the starting pulse of the first burst signal BS1 is a positive first pulse PL1, and the starting pulse of the second burst signal BS2 is a negative second pulse PL2. As shown in FIG. 3B, a starting pulse of the first burst signal BS1 may alternatively have a polarity the same as that of a starting pulse of the second burst signal BS2. Specifically, the starting pulse of the first burst signal BS1 is a positive first pulse PL1, and the starting pulse of the second burst signal BS2 is also a positive first pulse PL1.

In some embodiments, the first burst signal BS1 and the second burst signal BS2 occur alternately in timing. For example, a manner of arranging the first burst signal BS1 and the second burst signal BS2 in timing is “first burst signal BS1, second burst signal BS2, first burst signal BS1, second burst signal BS2, . . . ”. As shown in FIG. 2, in the first embodiment, when the first burst signal BS1 and the second burst signal BS2 are monophasic burst signals with opposite polarities, the first burst signal BS1 and the second burst signal BS2 that occur alternately can implement a biphasic charge balance, to avoid damage caused by the electrical stimulation signal ES to a tissue of an organism as much as possible. Different from the first embodiment, as shown in FIG. 3A and FIG. 3B, in the second embodiment and the third embodiment, when the first burst signal BS1 and the second burst signal BS2 are biphasic burst signals, the first pulses PL1 and the second pulses PL2 occurring alternately in each of the first burst signal BS1 and the second burst signal BS2 can implement a biphasic charge balance, to avoid damage caused by the electrical stimulation signal ES to a tissue of an organism as much as possible.

As shown in FIG. 2, in some embodiments, in addition to occurring alternately in timing, the first burst signal BS1 and the second burst signal BS2 with opposite polarities (namely, the first burst signal BS1 and the second burst signal BS2 are monophasic burst signals with opposite polarities) are further spaced apart by a period of time (namely, interval time T). For example, there is an interval time T between the first burst signal BS1 and the second burst signal BS2 adjacent to each other. In this embodiment, a delayed biphasic charge balance may be implemented, to further avoid damage caused by the electrical stimulation signal ES to a tissue of an organism as much as possible. In some embodiments, the interval time T between the first burst signal BS1 and the second burst signal BS2 ranges from 0 seconds to 5 seconds. In a preferred embodiment, the interval time T ranges from 0 seconds to 4.9995 seconds. In some embodiments, similar to FIG. 2 in which the first burst signal BS1 and the second burst signal BS2 are each implemented by a monophasic burst signal, in FIG. 3A and FIG. 3B, the first burst signal BS1 and the second burst signal BS2 implemented by biphasic burst signals may also be spaced apart from each other by a period of time (namely, interval time T′), and the period of time ranges from 0 seconds to 5 seconds. In addition, in a preferred embodiment, the interval time T′ ranges from 0 seconds to 4.9995 seconds.

In some embodiments, at a high frequency, duration of each pulse cycle is short. If the pulse frequency is 500 thousand Hz, duration of the pulse cycle is 2 microseconds. When each of the first burst signal BS1 and the second burst signal BS2 is a biphasic burst signal, it is possible that the control circuit 20 implemented by the low-performance operational circuit cannot quickly switch between positive and negative phases to enable the electrical stimulation circuit 40 to correctly generate the electrical stimulation signal ES. Specifically, after the control circuit 20 implemented by the low-performance operational circuit emits an instruction to control the electrical stimulation circuit 40 to generate a positive pulse (for example, the first pulse PL1), the control circuit 20 needs to process other interrupt requests of the electrical stimulation device 10, and because of low operational performance, the control circuit 20 cannot send, when the pulse cycle (for example, the first pulse cycle Tsi) of the positive pulse ends or after a period of time elapsing since the pulse cycle of the positive pulse ends, an instruction in time to control the electrical stimulation circuit 40 to generate a negative pulse (for example, the second pulse PL2). Therefore, by implementing the first burst signal BS1 and the second burst signal BS2 using monophasic burst signals with opposite polarities, the control circuit 20 does not need to perform quick changeover between positive and negative phases, thereby ensuring that the control circuit 20 implemented by a high-performance operational circuit or a low-performance operational circuit can control the electrical stimulation circuit 40 to correctly generate the electrical stimulation signal ES.

In some embodiments, the power supply of the power supply circuit 30 may be implemented by a battery, such as a button battery or a lithium battery. Compared with a power supply in which the utility power is directly converted into a direct-current power source, the battery has a low power capacity, provides a low power, and has a small volume. In this embodiment, through the power supply circuit 30 with a small volume, the electrical stimulation device 10 can be miniaturized, thereby making it convenient to carry the electrical stimulation device 10. Furthermore, based on the low power consumption of the electrical stimulation device 10, the quantity of times of replacing the battery of the power supply circuit 30 can be reduced. In some embodiments, when the power supply circuit 30 is implemented by a button battery, the first power supply signal VDD has a power ranging from 0.1 milliwatts to 30 milliwatts. In some other embodiments, when the power supply circuit 30 is implemented by a lithium battery, the first power supply signal VDD has a power ranging from 0.1 milliwatts to 30 watts. That is to say, the power that the power supply circuit 30 implemented by the lithium battery can provide may be higher than the power that the power supply circuit 30 implemented by the button battery can provide.

FIG. 4 is a schematic diagram of an electrical stimulation device 10 according to some embodiments of the disclosure. In some embodiments, an electrical stimulation circuit 40 includes a boost circuit 41. The boost circuit 41 is coupled to a control circuit 20 and a power supply circuit 30. The boost circuit 41 is controlled by the control circuit 20 to boost a first power supply signal VDD to generate a second power supply signal HV. In some embodiments, when the power supply circuit 30 is implemented by a button battery, the second power supply signal HV has a voltage ranging from 0.1 volts (V) to 100 volts. In some other embodiments, when the power supply circuit 30 is implemented by a lithium battery, the second power supply signal HV has a voltage ranging from 0.1 volts to 200 volts. That is to say, the voltage of the second power supply signal HV for the power supply circuit 30 implemented by the lithium battery that is obtained through conversion of the boost circuit 41 may be higher than the voltage of the second power supply signal HV for the power supply circuit 30 implemented by the button battery that is obtained through conversion of the boost circuit 41. In some embodiments, when a first burst signal BS1 and a second burst signal BS2 are monophasic burst signals with opposite polarities, a first pulse PL1 and a second pulse PL2 may be formed by the second power supply signal HV with a low voltage (for example, as shown in FIG. 2, a peak voltage of the first pulse PL1 is positive 20 volts, and a peak voltage of the second pulse PL2 is negative 20 volts). Therefore, in this situation, the power supply circuit 30 may be implemented by a button battery. In some other embodiments, when each of the first burst signal BS1 and the second burst signal BS2 is a biphasic burst signal, a first pulse PL1 and a second pulse PL2 may be formed by the second power supply signal HV with a high voltage (for example, as shown in FIG. 3A and FIG. 3B, a peak voltage of the first pulse PL1 is positive 30 volts, and a peak voltage of the second pulse PL2 is negative 30 volts). Therefore, in this situation, the power supply circuit 30 may be implemented by a lithium battery.

As shown in FIG. 4, in some embodiments, the electrical stimulation device 10 further includes at least one first electrode 50A and at least one second electrode 50B that are coupled to the electrical stimulation circuit 40. The first electrode 50A and the second electrode 50B may be an electrode pair and have opposite polarities. For example, the first electrode 50A is a positive electrode, and the second electrode 50B is a negative electrode. However, the disclosure is not limited thereto. The first electrode 50A may be a negative electrode, and the second electrode 50B may be a positive electrode. The first electrode 50A and the second electrode 50B may be arranged in a region (hereinafter referred to as an arrangement region) of an organism, which is close to a to-be-stimulated target region of the organism, to electrically stimulate the target region. Referring to FIG. 1B together again, an example in which the to-be-stimulated target region is a dorsal penile nerve is taken, and the first electrode 50A and the second electrode 50B (the electrode pair 11 shown in FIG. 1B) can be arranged between the middle of a human penis (namely, reproductive organ P) and the corona of glans penis (namely, the arrangement region). An electrical stimulation signal ES is output by the electrical stimulation circuit 40 to a certain electrode, for example, the first electrode 50A (or the second electrode 50B), and is conducted through a tissue of the human body and then returned to the electrical stimulation circuit 40 by the other electrode, for example, the second electrode 50B (or the first electrode 50A), to complete electrical stimulation on the target region. In some embodiments, the target region for an electrical stimulation may alternatively receive small energy or a small quantity of charges in a unit time, thereby ensuring that the electrical stimulation is an electrical stimulation with a subthreshold. In this embodiment, a possibility that the user senses paresthesia can be reduced, and even a paresthesia-free electrical stimulation without sensation is achieved.

In addition, because a usual high-frequency electrical stimulation (ranging from 1000 Hz to 10 million Hz) can achieve a paresthesia-free electrical stimulation without sensation, the user is prone to doubt whether the user is undergoing an electrical stimulation when the user is undergoing the electrical stimulation, and even therefore performs startup or shutdown on the electrical stimulation device 10 again, to interrupt original running of the electrical stimulation of the electrical stimulation device 10. Therefore, it is also proposed in this embodiment that by accumulating charges of the monophasic burst signal to a specific threshold (before being charge-balanced), the user can feel slight beating sensation, and the quantity of accumulated charges may also be referred to as a minimum sensible quantity of charges.

Because paresthesia may be very subjective, the slight beating sensation caused by the electrical stimulation may cause no uncomfortable feeling or discomfort to an ordinary person. Therefore, instead, without causing the user to feel uncomfortable or within a tolerable range, a tiny feeling caused by the electrical stimulation can be used as a feeling of relief for confirming that the electrical stimulation is running, to enable the user to achieve a better use experience. An example of calculation about the minimum sensible quantity of charges (Qₘᵢₙ) is as follows: The high-frequency electrical stimulation frequency (namely, the first pulse frequency or the second pulse frequency) generated by the electrical stimulation device 10 of the disclosure can be represented by 500 thousand Hz, to stimulate a tissue of the organism. As shown in Formula 10 and referring to FIG. 2 together, it is assumed that the target region of the tissue of the organism that is electrically stimulated has an impedance value (Rₜᵢₛₛᵤₑ) of 500 ohm and an absolute value of an output voltage (namely, peak voltage) (Vₒᵤₜ) of the first pulse PL1 or the second pulse PL2 is 12.6 volts, it may be learned that the first pulse PL1 or the second pulse PL2 has a current value (Iₜᵢₛₛᵤₑ) of 25.2 microamp. When the first pulse frequency of the first pulse PL1 is 500 thousand Hz, the first burst width T_{d1} is 10 milliseconds, the first pulse cycle Tₛ₁ is 2 microseconds, and the first pulse width Tₑ₁ of the first pulse PL1 may be 0.18 microseconds, it may be learned that the minimum sensible quantity of charges (Qₘᵢₙ) is 22.7 microcoulombs (μC). That is to say, before being charge-balanced, monophasic charges accumulated by the monophasic burst signal (namely, the first monophasic burst signal or the second monophasic burst signal), that is, the minimum sensible quantity of charges (Qₘᵢₙ) is 22.7 μC, and then an impedance value difference between human bodies is consider, so that the minimum sensible quantity of charges (Qₘᵢₙ) is set to about 20 μC. As long as the monophasic charges accumulated by the monophasic burst signal is greater than this minimum sensible quantity of charges (Qₘᵢₙ), the electrical stimulation is an electrical stimulation with sensation sensible by the user, to enable the user to learn that the electrical stimulation is being performed.

$$Q min = \frac{{V}_{out}}{{R}_{tissue}} \times {T}_{e 1} \times \frac{{T}_{d 1}}{{T}_{s 1}} \qquad ( Formula 10 )$$

As shown in FIG. 4, in some embodiments, the electrical stimulation circuit 40 further includes a first output circuit 43 and a second output circuit 45. The first output circuit 43 and the second output circuit 45 are coupled to the boost circuit 41, the control circuit 20, the first electrode 50A, and the second electrode 50B. The first output circuit 43 is controlled by the control circuit 20 to generate the first pulses PL1 at the first electrode 50A and the second electrode 50B according to the second power supply signal HV. The second output circuit 45 is controlled by the control circuit 20 to generate the second pulses PL2 at the first electrode 50A and the second electrode 50B according to the second power supply signal HV.

As shown in FIG. 4, in some embodiments, the first output circuit 43 includes a first driving circuit 431, a first pulse circuit 433, and a second pulse circuit 435. The first driving circuit 431 is coupled to the control circuit 20. The first pulse circuit 433 is coupled to the boost circuit 41, the first driving circuit 431, and the first electrode 50A. The second pulse circuit 435 is coupled to the first driving circuit 431, the second electrode 50B, and a reference ground terminal 60. The first driving circuit 431 is controlled by the control circuit 20 to emit a first driving signal DV1. The first pulse circuit 433 receives the second power supply signal HV according to the first driving signal DV1 and transmits the second power supply signal HV to the first electrode 50A. The second pulse circuit 435 transmits the second power supply signal HV from the second electrode 50B to the reference ground terminal 60 according to the first driving signal DV1. The control circuit 20 controls, according to the first pulse frequency and the first pulse duty cycle of the first pulses PL1, the first driving circuit 431 to emit the first driving signal DV1, to enable the first output circuit 43 to generate the first pulses PL1 at the first electrode 50A and the second electrode 50B.

For example, the control circuit 20 changes, according to a setting parameter (hereinafter referred to as a first setting parameter) corresponding to the first pulse frequency of the first pulses PL1 and a setting parameter (hereinafter referred to as a second setting parameter) corresponding to the first pulse duty cycle of the first pulses PL1 that are stored in the memory thereof or according to the first setting parameter corresponding to the first pulse frequency of the first pulses PL1 and the second setting parameter corresponding to the first pulse duty cycle of the first pulses PL1 that are input after the user operates the user interface, a driving frequency (hereinafter referred to as a first driving frequency) the first driving signal DV1 emitted by the first driving circuit 431. The first pulse circuit 433 changes, according to the first driving frequency of the first driving signal DV1, a switching frequency thereof (namely, a frequency of switching between on and off) (hereinafter referred to as a first switching frequency). The second pulse circuit 435 changes, according to the first driving frequency of the first driving signal DV1, a switching frequency thereof (namely, a frequency of switching between on and off) (hereinafter referred to as a second switching frequency). That is to say, the first pulse circuit 433 and the second pulse circuit 435 have synchronous switching frequencies (namely, the first switching frequency and the second switching frequency are synchronous). When the first pulse circuit 433 and the second pulse circuit 435 are on, the first pulse circuit 433 receives the second power supply signal HV, and transmits the second power supply signal HV to the first electrode 50A. When the first electrode 50A and the second electrode 50B are arranged in the arrangement region, the second power supply signal HV received by the first electrode 50A is transmitted to the second electrode 50B through the arrangement region. The second pulse circuit 435 transmits the second power supply signal HV from the second electrode 50B to the reference ground terminal 60, to serve as the first pulse PL1. In this embodiment, the second power supply signal HV may be converted into the first pulse PL1. When the first pulse circuit 433 and the second pulse circuit 435 are off, the first pulse circuit 433 does not transmit the second power supply signal HV to the first electrode 50A, and therefore the second pulse circuit 435 does not receive the second power supply signal HV from the second electrode 50B. The control circuit 20 repeatedly enables the first pulse circuit 433 and the second pulse circuit 435 to be on and off through the first driving signal DV1 of the first driving circuit 431, to enable the first output circuit 43 to generate a plurality of cyclical first pulses PL1. The control circuit 20 can control changes in the first pulse frequency and the first pulse duty cycle of the first pulse PL1. Specifically, the control circuit 20 changes the first driving frequency, the first switching frequency, and the second switching frequency according to the first setting parameter and the second setting parameter, and therefore changes the first pulse frequency and the first pulse duty cycle of the first pulse PL1.

As shown in FIG. 4, in some embodiments, similar to the first output circuit 43, the second output circuit 45 includes a second driving circuit 451, a third pulse circuit 453, and a fourth pulse circuit 455. The second driving circuit 451 is coupled to the control circuit 20. The second driving circuit 451 is controlled by the control circuit 20 to emit a second driving signal DV2. Different from the first output circuit 43, in the second output circuit 45, the third pulse circuit 453 is coupled to the boost circuit 41, the second driving circuit 451, and the second electrode 50B. The fourth pulse circuit 455 is coupled to the second driving circuit 451, the first electrode 50A, and the reference ground terminal 60. The third pulse circuit 453 receives the second power supply signal HV according to the second driving signal DV2 and transmits the second power supply signal HV to the second electrode 50B. The fourth pulse circuit 455 transmits the second power supply signal HV from the first electrode 50A to the reference ground terminal according to the second driving signal DV2. The control circuit 20 controls, according to the second pulse frequency and the second pulse duty cycle of the second pulses PL2, the second driving circuit 451 to emit the second driving signal DV2, to enable the second output circuit 45 to generate the second pulses PL2 at the first electrode 50A and the second electrode 50B.

For example, the control circuit 20 changes, according to a setting parameter (hereinafter referred to as a third setting parameter) corresponding to the second pulse frequency of the second pulses PL2 and a setting parameter (hereinafter referred to as a fourth setting parameter) corresponding to the second pulse duty cycle of the second pulses PL2 that are stored in the memory thereof or according to the third setting parameter corresponding to the second pulse frequency of the second pulses PL2 and the fourth setting parameter corresponding to the second pulse duty cycle of the second pulses PL2 that are input after the user operates the user interface, a driving frequency (hereinafter referred to as a second driving frequency) the second driving signal DV2 emitted by the second driving circuit 451. The third pulse circuit 453 changes, according to the second driving frequency of the second driving signal DV2, a switching frequency thereof (namely, a frequency of switching between on and off) (hereinafter referred to as a third switching frequency). The fourth pulse circuit 455 changes, according to the second driving frequency of the second driving signal DV2, a switching frequency thereof (namely, a frequency of switching between on and off) (hereinafter referred to as a fourth switching frequency). That is to say, the third pulse circuit 453 and the fourth pulse circuit 455 have synchronous switching frequencies (namely, the third switching frequency and the fourth switching frequency are synchronous). When the third pulse circuit 453 and the fourth pulse circuit 455 are on, the third pulse circuit 453 receives the second power supply signal HV, and transmits the second power supply signal HV to the second electrode 50B. When the first electrode 50A and the second electrode 50B are arranged in the arrangement region, the second power supply signal HV received by the second electrode 50B is transmitted to the first electrode 50A through the arrangement region. The fourth pulse circuit 455 transmits the second power supply signal HV from the first electrode 50A to the reference ground terminal 60, to serve as the second pulse PL2. In this embodiment, the second power supply signal HV may be converted into the second pulse PL2. When the third pulse circuit 453 and the fourth pulse circuit 455 are off, the third pulse circuit 453 does not transmit the second power supply signal HV to the second electrode 50B, and therefore the fourth pulse circuit 455 does not receive the second power supply signal HV from the first electrode 50A. The control circuit 20 repeatedly enables the third pulse circuit 453 and the fourth pulse circuit 455 to be on and off through the second driving signal DV2 of the second driving circuit 451, to enable the second output circuit 45 to generate a plurality of cyclical second pulses PL2. The control circuit 20 can control changes in the second pulse frequency and the second pulse duty cycle of the second pulse PL2. Specifically, the control circuit 20 changes the second driving frequency, the third switching frequency, and the fourth switching frequency according to the third setting parameter and the fourth setting parameter, and therefore changes the second pulse frequency and the second pulse duty cycle of the second pulse PL2.

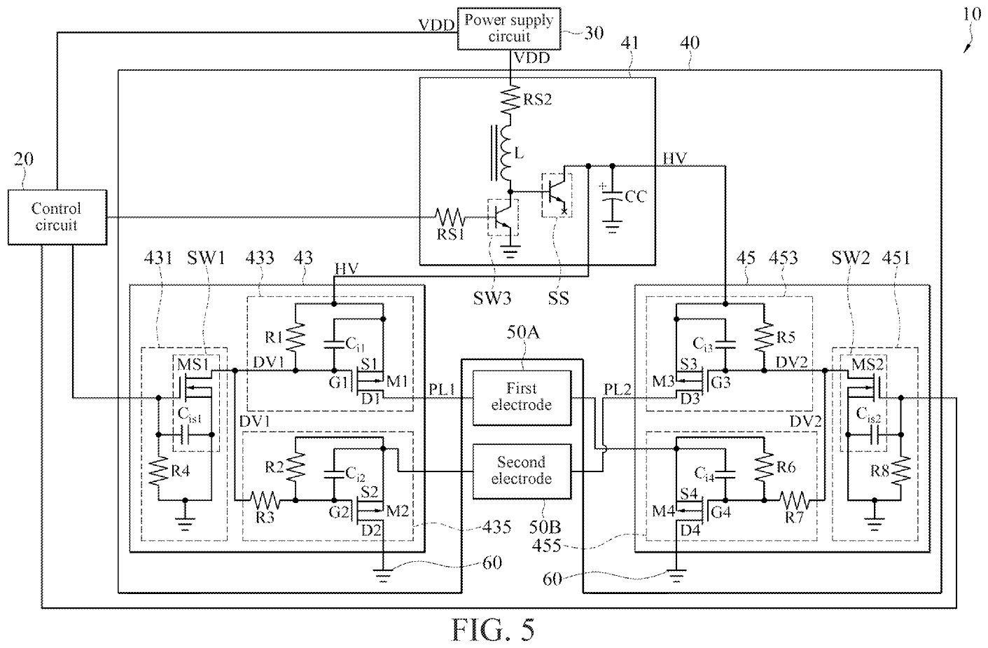

FIG. 5 is a detailed schematic circuit diagram of an electrical stimulation circuit 40 according to some embodiments of the disclosure. In some embodiments, a first pulse circuit 433 includes a first transistor M1 having a first input capacitor Cᵢ₁ and a first resistor R1. The first input capacitor Cᵢ₁ is an internal capacitor of the first transistor M1. The first resistor R1 is connected in parallel to the first input capacitor Cᵢ₁. The first transistor M1 includes a first control terminal G1, a first input terminal S1, and a first output terminal D1. The first input capacitor Cᵢ₁ is between the first control terminal G1 and the first input terminal S1. The first control terminal G1 is coupled to a first driving circuit 431, the first input terminal S1 is coupled to a boost circuit 41, and the first output terminal D1 is coupled to a first electrode 50A. The first control terminal G1 receives a first driving signal DV1, the first input terminal S1 receives a second power supply signal HV, and the first output terminal D1 transmits the second power supply signal HV to the first electrode 50A. In some embodiments, the first transistor M1 is a field effect transistor, to conform to the first pulse frequency of the first pulse PL1.

For example, the first transistor M1 may be a P-type transistor, the first control terminal G1 is a gate terminal, the first input terminal S1 is a source terminal, and the first output terminal D1 is a drain terminal. When the first transistor M1 is on according to the first driving signal DV1 received by the first control terminal G1, the first transistor M1 receives the second power supply signal HV through the first input terminal S1, and transmits the second power supply signal HV to the first electrode 50A through the first output terminal D1. When the first transistor M1 is off according to the first driving signal DV1 received by the first control terminal G1, the first transistor M1 does not transmit the second power supply signal HV to the first electrode 50A.

In some embodiments, a product of a resistance value (hereinafter referred to as a first resistance value) of the first resistor R1 and a capacitance value (hereinafter referred to as a first capacitance value) of the first input capacitor Cᵢ₁ is less than half of the first pulse width Tₑ₁ of each first pulse PL1. As shown in Formula 11, R1ᵥ is the first resistance value, and Cᵢ₁ᵥ is the first capacitance value. In this embodiment, it is ensured that the first pulse PL1 can be correctly output. For example, when the first pulse frequency of the first pulse PL1 is 500 thousand Hz, the first pulse width Tₑ₁ of the first pulse PL1 may be 0.18 microseconds, half of the first pulse width Tₑ₁ is 0.9 microseconds, the first capacitance value of the first input capacitor Cᵢ₁ may be 10 picofarads (pF), the first resistance value of the first resistor R1 may be 3 ohms (Ω), and the product of the first capacitance value and the first resistance value is 30×10⁻¹² and is less than 0.9×10⁻⁶. In this embodiment, it is ensured that after the first transistor M1 is on and transmits the second power supply signal HV to the first electrode 50A (namely, after the electrical stimulation circuit 40 emits one first pulse PL1), when the electrical stimulation circuit 40 intends to emit a next first pulse PL1, the first input capacitor Cᵢ₁ has been successfully fully discharged, to enable the first transistor M1 to be on again. In some embodiments, the first capacitance value of the first input capacitor Cᵢ₁ may be in a negative correlation with the first pulse frequency of the first pulse PL1. For example, when the first pulse frequency of the first pulse PL1 is 200 thousand Hz, the first capacitance value of the first input capacitor Cᵢ₁ may be 20 picofarads; when the first pulse frequency of the first pulse PL1 is 500 thousand Hz, the first capacitance value of the first input capacitor Cᵢ₁ may be 10 picofarads; and when the first pulse frequency of the first pulse PL1 is 1 million Hz, the first capacitance value of the first input capacitor Cᵢ₁ may be 5 picofarads.

$$R {1}_{v} \times {C}_{i 1 v} \leq \frac{1}{2} \times {T}_{e 1} \qquad ( Formula 11 )$$

As shown in FIG. 5, in some embodiments, the second pulse circuit 435 includes a second transistor M2 having a second input capacitor Cᵢ₂, a second resistor R2, and a third resistor R3. The second input capacitor Cᵢ₂ is an internal capacitor of the second transistor M2. The second resistor R2 is connected in parallel to the second input capacitor Cᵢ₂, and configured to prevent a leakage current of the second transistor M2. The second transistor M2 includes a second control terminal G2, a second input terminal S2, and a second output terminal D2. The third resistor R3 is between the first driving circuit 431 and the second control terminal G2. The second input capacitor Cᵢ₂ is between the second control terminal G2 and the second input terminal S2. The second control terminal G2 is coupled to the first driving circuit 431, the second input terminal S2 is coupled to the second electrode 50B, and the second output terminal D2 is coupled to the reference ground terminal 60. The second control terminal G2 receives the first driving signal DV1, the second input terminal S2 receives the second power supply signal HV from the second electrode 50B, and the second output terminal D2 transmits the second power supply signal HV to the reference ground terminal 60. In some embodiments, the second transistor M2 is a field effect transistor, to conform to the first pulse frequency of the first pulse PL1.

For example, the second transistor M2 may be a P-type transistor, the second control terminal G2 is a gate terminal, the second input terminal S2 is a source terminal, and the second output terminal D2 is a drain terminal. When the first electrode 50A and the second electrode 50B are arranged in the arrangement region and the first electrode 50A receives the second power supply signal HV, the second power supply signal HV received by the first electrode 50A is transmitted to the second electrode 50B through the arrangement region. When the second transistor M2 is on according to the first driving signal DV1 received by the second control terminal G2, the second transistor M2 receives the second power supply signal HV from the second electrode 50B through the second input terminal S2, and transmits the second power supply signal HV to the reference ground terminal 60 through the second output terminal D2, to serve as the first pulse PL1. When the second transistor M2 is off according to the first driving signal DV1 received by the second control terminal G2, the second transistor M2 does not transmit the second power supply signal HV to the reference ground terminal 60.

In some embodiments, similar to the first resistance value of the first resistor R1 and the first capacitance value of the first input capacitor Cᵢ₁, a product of a resistance value (hereinafter referred to as a third resistance value) of the third resistor R3 and a capacitance value (hereinafter referred to as a second capacitance value) of the second input capacitor Cᵢ₂ is less than half of the first pulse width Tₑ₁ of each first pulse PL1. As shown in Formula 12, R3ᵥ is the third resistance value, and Cᵢ₂ᵥ is the second capacitance value. In this embodiment, it is ensured that the first pulse PL1 can be correctly output. For example, when the first pulse frequency of the first pulse PL1 is 500 thousand Hz, the first pulse width Tₑ₁ of the first pulse PL1 may be 0.18 microseconds, half of the first pulse width Tₑ₁ is 0.9 microseconds, the second capacitance value of the second input capacitor Cᵢ₂ may be 10 picofarads, the third resistance value of the third resistor R3 may be 3 ohms, and the product of the second capacitance value and the third resistance value is 30×10⁻¹² and is less than 0.9×10⁻⁶. In this embodiment, it is ensured that after the second transistor M2 is on and transmits the second power supply signal HV to the reference ground terminal 60 (namely, after the electrical stimulation circuit 40 emits one first pulse PL1), when the electrical stimulation circuit 40 intends to emit a next first pulse PL1, the second input capacitor Cᵢ₂ has been successfully fully discharged, to enable the second transistor M2 to be on again. In some embodiments, similar to the first capacitance value of the first input capacitor Cᵢ₁, the second capacitance value of the second input capacitor Cᵢ₂ may be in a negative correlation with the first pulse frequency of the first pulse PL1. For example, when the first pulse frequency of the first pulse PL1 is 200 thousand Hz, the second capacitance value of the second input capacitor Cᵢ₂ may be 20 picofarads; when the first pulse frequency of the first pulse PL1 is 500 thousand Hz, the second capacitance value of the second input capacitor Cᵢ₂ may be 10 picofarads; and when the first pulse frequency of the first pulse PL1 is 1 million Hz, the second capacitance value of the second input capacitor Cᵢ₂ may be 5 picofarads.

$$R {3}_{v} \times {C}_{i 2 v} \leq \frac{1}{2} \times {T}_{e 1} \qquad ( Formula 12 )$$

As shown in FIG. 5, in some embodiments, the first driving circuit 431 includes a first changeover switch SW1 and a fourth resistor R4. The fourth resistor R4 is between the first changeover switch SW1 and the reference ground terminal 60. The fourth resistor R4 is configured to prevent a leakage current of the first changeover switch SW1. The first changeover switch SW1 is coupled to the control circuit 20, the reference ground terminal 60, the first control terminal G1 of the first transistor M1 of the first pulse circuit 433, and the second control terminal G2 of the second transistor M2 of the second pulse circuit 435. The control circuit 20 switches a changeover state of the first changeover switch SW1 according to the first setting parameter corresponding to the first pulse frequency of the first pulse PL1 and the second setting parameter corresponding to the first pulse duty cycle of the first pulse PL1, to control the first driving circuit 431 to emit the first driving signal DV1. For example, the control circuit 20 repeatedly switches the changeover state of the first changeover switch SW1 between on and off according to the first setting parameter corresponding to the first pulse frequency of the first pulse PL1 and the second setting parameter corresponding to the first pulse duty cycle of the first pulse PL1, to control the first driving circuit 431 to emit the first driving signal DV1 and change the first driving frequency of the first driving signal DV1. In some embodiments, the first changeover switch SW1 may be implemented by an electronic switch. The electronic switch is, for example, a transistor (such as a fifth transistor MS1). In some embodiments, the fifth transistor MS1 may be a field effect transistor, to conform to the first pulse frequency of the first pulse PL1, and the fifth transistor MS1 may have a first internal capacitor Cᵢₛ₁ connected in parallel to the fourth resistor R4. In some embodiments, a capacitance value of the first internal capacitor Cᵢₛ₁ may be in a negative correlation with the first pulse frequency of the first pulse PL1. For example, when the first pulse frequency of the first pulse PL1 is 200 thousand Hz, the capacitance value of the first internal capacitor Cᵢₛ₁ may be 20 picofarads; when the first pulse frequency of the first pulse PL1 is 500 thousand Hz, the capacitance value of the first internal capacitor Cᵢₛ₁ may be 10 picofarads; and when the first pulse frequency of the first pulse PL1 is 1 million Hz, the capacitance value of the first internal capacitor Cᵢₛ₁ may be 5 picofarads.

As shown in FIG. 5, in some embodiments, similar to the first pulse circuit 433, the third pulse circuit 453 includes a third transistor M3 having a third input capacitor Cᵢ₃ and a fifth resistor R5. The third input capacitor Cᵢ₃ is an internal capacitor of the third transistor M3. The fifth resistor R5 is connected in parallel to the third input capacitor Cᵢ₃. The third transistor M3 includes a third control terminal G3, a third input terminal S3, and a third output terminal D3. The third input capacitor Cᵢ₃ is between the third control terminal G3 and the third input terminal S3. The third control terminal G3 is coupled to the second driving circuit 451, and the third input terminal S3 is coupled to the boost circuit 41. The third control terminal G3 receives the second driving signal DV2, and the third input terminal S3 receives the second power supply signal HV. Different from the first pulse circuit 433, in the third pulse circuit 453, the third output terminal D3 is coupled to the second electrode 50B, and the third output terminal D3 transmits the second power supply signal HV to the second electrode 50B. In some embodiments, the third transistor M3 is a field effect transistor, to conform to the second pulse frequency of the second pulse PL2.

For example, the third transistor M3 may be a P-type transistor, the third control terminal G3 is a gate terminal, the third input terminal S3 is a source terminal, and the third output terminal D3 is a drain terminal. When the third transistor M3 is on according to the second driving signal DV2 received by the third control terminal G3, the third transistor M3 receives the second power supply signal HV through the third input terminal S3, and transmits the second power supply signal HV to the second electrode 50B through the third output terminal D3. When the third transistor M3 is off according to the second driving signal DV2 received by the third control terminal G3, the third transistor M3 does not transmit the second power supply signal HV to the second electrode 50B.

In some embodiments, a product of a resistance value (hereinafter referred to as a fifth resistance value) of the fifth resistor R5 and a capacitance value (hereinafter referred to as a third capacitance value) of the third input capacitor Cᵢ₃ is less than half of the second pulse width Tₑ₂ of each second pulse PL2. As shown in Formula 13, R5ᵥ is the fifth resistance value, and Cᵢ₃ᵥ is the third capacitance value. In this embodiment, it is ensured that the second pulse PL2 can be correctly output. For example, when the second pulse frequency of the second pulse PL2 is 500 thousand Hz, the second pulse width Tₑ₂ of the second pulse PL2 may be 0.18 microseconds, half of the second pulse width Tₑ₂ is 0.9 microseconds, the third capacitance value of the third input capacitor Cᵢ₃ may be 10 picofarads, the fifth resistance value of the fifth resistor R5 may be 3 ohms, and the product of the third capacitance value and the fifth resistance value is 30×10⁻¹² and is less than 0.9×10⁻⁶. In this embodiment, it is ensured that after the third transistor M3 is on and transmits the second power supply signal HV to the second electrode 50B (namely, after the electrical stimulation circuit 40 emits one second pulse PL2), when the electrical stimulation circuit 40 intends to emit a next second pulse PL2, the third input capacitor Cᵢ₃ has been successfully fully discharged, to enable the third transistor M3 to be on again. In some embodiments, the third capacitance value of the third input capacitor Cᵢ₃ may be in a negative correlation with the second pulse frequency of the second pulse PL2. For example, when the second pulse frequency of the second pulse PL2 is 200 thousand Hz, the third capacitance value of the third input capacitor Cᵢ₃ may be 20 picofarads; when the second pulse frequency of the second pulse PL2 is 500 thousand Hz, the third capacitance value of the third input capacitor Cᵢ₃ may be 10 picofarads; and when the second pulse frequency of the second pulse PL2 is 1 million Hz, the third capacitance value of the third input capacitor Cᵢ₃ may be 5 picofarads.

$$R {5}_{v} \times {C}_{i 3 v} \leq \frac{1}{2} \times {T}_{e 2} \qquad ( Formula 13 )$$

As shown in FIG. 5, in some embodiments, similar to the second pulse circuit 435, the fourth pulse circuit 455 includes a fourth transistor M4 having a fourth input capacitor Cᵢ₄, a sixth resistor R6, and a seventh resistor R7. The fourth input capacitor Cᵢ₄ is an internal capacitor of the fourth transistor M4. The sixth resistor R6 is connected in parallel to the fourth input capacitor Cᵢ₄, and configured to prevent a leakage current of the fourth transistor M4. The fourth transistor M4 includes a fourth control terminal G4, a fourth input terminal S4, and a fourth output terminal D4. The seventh resistor R7 is between the second driving circuit 451 and the fourth control terminal G4. The fourth input capacitor Cᵢ₄ is between the fourth control terminal G4 and the fourth input terminal S4. The fourth control terminal G4 is coupled to the second driving circuit 451, and the fourth output terminal D4 is coupled to the reference ground terminal 60. The fourth control terminal G4 receives the second driving signal DV2, and the fourth output terminal D4 transmits the second power supply signal HV to the reference ground terminal 60. Different from the second pulse circuit 435, in the fourth pulse circuit 455, the fourth input terminal S4 is coupled to the first electrode 50A, and the fourth input terminal S4 receives the second power supply signal HV from the first electrode 50A. In some embodiments, the fourth transistor M4 is a field effect transistor, to conform to the second pulse frequency of the second pulse PL2.

For example, the fourth transistor M4 may be a P-type transistor, the fourth control terminal G4 is a gate terminal, the fourth input terminal S4 is a source terminal, and the fourth output terminal D4 is a drain terminal. When the first electrode 50A and the second electrode 50B are arranged in the arrangement region and the first electrode 50A receives the second power supply signal HV, the second power supply signal HV received by the second electrode 50B is transmitted to the first electrode 50A through the arrangement region. When the fourth transistor M4 is on according to the second driving signal DV2 received by the fourth control terminal G4, the fourth transistor M4 receives the second power supply signal HV from the first electrode 50A through the fourth input terminal S4, and transmits the second power supply signal HV to the reference ground terminal 60 through the fourth output terminal D4, to serve as the second pulse PL2. When the fourth transistor M4 is off according to the second driving signal DV2 received by the fourth control terminal G4, the fourth transistor M4 does not transmit the second power supply signal HV to the reference ground terminal 60.

In some embodiments, similar to the fifth resistance value of the fifth resistor R5 and the third capacitance value of the third input capacitor Cᵢ₃, a product of a resistance value (hereinafter referred to as a seventh resistance value) of the seventh resistor R7 and a capacitance value (hereinafter referred to as a fourth capacitance value) of the fourth input capacitor Cᵢ₄ is less than half of the second pulse width Tₑ₂ of each second pulse PL2. As shown in Formula 14, R7ᵥ is the seventh resistance value, and Cᵢ₄ᵥ is the fourth capacitance value. In this embodiment, it is ensured that the second pulse PL2 can be correctly output. For example, when the second pulse frequency of the second pulse PL2 is 500 thousand Hz, the second pulse width Tₑ₂ of the second pulse PL2 may be 0.18 microseconds, half of the second pulse width Tₑ₂ is 0.9 microseconds, the fourth capacitance value of the fourth input capacitor Cᵢ₄ may be 10 picofarads, the seventh resistance value of the seventh resistor R7 may be 3 ohms, and the product of the fourth capacitance value and the seventh resistance value is 30×10⁻¹² and is less than 0.9×10⁻⁶. In this embodiment, it is ensured that after the fourth transistor M4 is on and transmits the second power supply signal HV to the reference ground terminal 60 (namely, after the electrical stimulation circuit 40 emits one second pulse PL2), when the electrical stimulation circuit 40 intends to emit a next second pulse PL2, the fourth input capacitor Cᵢ₄ has been successfully fully discharged, to enable the fourth transistor M4 to be on again. In some embodiments, similar to the third capacitance value of the third input capacitor Cᵢ₃, the fourth capacitance value of the fourth input capacitor Cᵢ₄ may be in a negative correlation with the second pulse frequency of the second pulse PL2. For example, when the second pulse frequency of the second pulse PL2 is 200 thousand Hz, the fourth capacitance value of the fourth input capacitor Cᵢ₄ may be 20 picofarads; when the second pulse frequency of the second pulse PL2 is 500 thousand Hz, the fourth capacitance value of the fourth input capacitor Cᵢ₄ may be 10 picofarads; and when the second pulse frequency of the second pulse PL2 is 1 million Hz, the fourth capacitance value of the fourth input capacitor Cᵢ₄ may be 5 picofarads.

$$R {7}_{v} \times {C}_{i 4 v} \leq \frac{1}{2} \times {T}_{e 2} \qquad ( Formula 14 )$$

As shown in FIG. 5, in some embodiments, similar to the first driving circuit 431, the second driving circuit 451 includes a second changeover switch SW2 and an eighth resistor R8. The eighth resistor R8 is between the second changeover switch SW2 and the reference ground terminal 60. The eighth resistor R8 is configured to prevent a leakage current of the second changeover switch SW2. The second changeover switch SW2 is coupled to the control circuit 20, the reference ground terminal 60, the third control terminal G3 of the third transistor M3 of the third pulse circuit 453, and the fourth control terminal G4 of the fourth transistor M4 of the fourth pulse circuit 455. The control circuit 20 switches a changeover state of the second changeover switch SW2 according to the third setting parameter corresponding to the second pulse frequency of the second pulse PL2 and the fourth setting parameter corresponding to the second pulse duty cycle of the second pulse PL2, to control the second driving circuit 451 to emit the second driving signal DV2. For example, the control circuit 20 repeatedly switches the changeover state of the second changeover switch SW2 between on and off according to the third setting parameter corresponding to the second pulse frequency of the second pulse PL2 and the fourth setting parameter corresponding to the second pulse duty cycle of the second pulse PL2, to control the second driving circuit 451 to emit the second driving signal DV2 and change the second driving frequency of the second driving signal DV2. In some embodiments, the second changeover switch SW2 may be implemented by an electronic switch. The electronic switch is, for example, a transistor (for example, a sixth transistor MS2). In some embodiments, the sixth transistor MS2 may be a field effect transistor, to conform to the second pulse frequency of the second pulse PL2, and the sixth transistor MS2 may have a second internal capacitor Cᵢₛ₂ connected in parallel to the eighth resistor R8. In some embodiments, a capacitance value of the second internal capacitor Cᵢₛ₂ may be in a negative correlation with the second pulse frequency of the second pulse PL2. For example, when the second pulse frequency of the second pulse PL2 is 200 thousand Hz, the capacitance value of the second internal capacitor Cᵢₛ₂ may be 20 picofarads; when the second pulse frequency of the second pulse PL2 is 500 thousand Hz, the capacitance value of the second internal capacitor Cᵢₛ₂ may be 10 picofarads; and when the second pulse frequency of the second pulse PL2 is 1 million Hz, the capacitance value of the second internal capacitor Cᵢₛ₂ may be 5 picofarads.

As shown in FIG. 5, in some embodiments, the boost circuit 41 includes a third changeover switch SW3, an inductor L, a charging capacitor CC, and a blocking switch SS. The third changeover switch SW3 is coupled to the control circuit 20, the inductor L, and the blocking switch SS, and the inductor L is coupled to the power supply circuit 30. The blocking switch SS is between a node between the third changeover switch SW3 and the inductor L and the charging capacitor CC, and configured to limit a flow direction of a signal (for example, a current). The control circuit 20 controls a changeover state of the third changeover switch SW3, namely, controls the third changeover switch SW3 to switch between on and off. The inductor L performs energy storage or release according to the changeover state of the third changeover switch SW3 and the first power supply signal VDD. Specifically, when the changeover state of the third changeover switch SW3 is on, the inductor L performs energy storage according to the first power supply signal VDD. When the changeover state of the third changeover switch SW3 is off, the inductor L performs energy release, and released energy is combined with the first power supply signal VDD, thereby generating the first charging signal to serve as the second power supply signal HV. The charging capacitor CC performs charging or discharging according to the changeover state of the third changeover switch SW3. Specifically, when the changeover state of the third changeover switch SW3 is off, the charging capacitor CC performs charging according to the first charging signal. When the changeover state of the third changeover switch SW3 is on, the charging capacitor CC performs discharging to generate the second charging signal, to serve as the second power supply signal HV. In this embodiment, the first power supply signal VDD may be boosted to the second power supply signal HV. In some embodiments, the third changeover switch SW3 may be implemented by an electronic switch, for example, a transistor (for example, a bipolar transistor). In some embodiments, the blocking switch SS may be implemented by a diode or a transistor.

In some embodiments, an inductance value of the inductor L is in a positive correlation with the first pulse frequency of the first pulse PL1 and the second pulse frequency of the second pulse PL2, and an impedance value of the inductor L is in a negative correlation with the first pulse frequency of the first pulse PL1 and the second pulse frequency of the second pulse PL2. For example, when the first pulse frequency of the first pulse PL1 (and/or the second pulse frequency of the second pulse PL2) is 200 thousand Hz, the inductance value of the inductor L may be 500 microhenries (H), and the impedance value of the inductor L may be 4.5 ohms; when the first pulse frequency of the first pulse PL1 (and/or the second pulse frequency of the second pulse PL2) is 500 thousand Hz, the inductance value of the inductor L may be 800 microhenries, and the impedance value of the inductor L may be 2 ohms; and when the first pulse frequency of the first pulse PL1 (and/or the second pulse frequency of the second pulse PL2) is 1 million Hz, the inductance value of the inductor L may be 2200 microhenries, and the impedance value of the inductor L may be 1 ohm. In some embodiments, a charging capacitance value of the charging capacitor CC is in a positive correlation with the first pulse frequency of the first pulse PL1 and the second pulse frequency of the second pulse PL2. For example, when the first pulse frequency of the first pulse PL1 (and/or the second pulse frequency of the second pulse PL2) is 200 thousand Hz, the charging capacitance value of the charging capacitor CC may be 47 microfarads; when the first pulse frequency of the first pulse PL1 (and/or the second pulse frequency of the second pulse PL2) is 500 thousand Hz, the charging capacitance value of the charging capacitor CC may be 100 microfarads; and when the first pulse frequency of the first pulse PL1 (and/or the second pulse frequency of the second pulse PL2) is 1 million Hz, the charging capacitance value of the charging capacitor CC may be 220 microfarads.

In some embodiments, the boost circuit 41 further includes two resistors (such as a ninth resistor RS1 and a tenth resistor RS2), located between the power supply circuit 30 and the inductor L and located between the control circuit 20 and the third changeover switch SW3 respectively and configured to limit a ceiling of a current to prevent the boost circuit 41 from being damaged due to an overload.

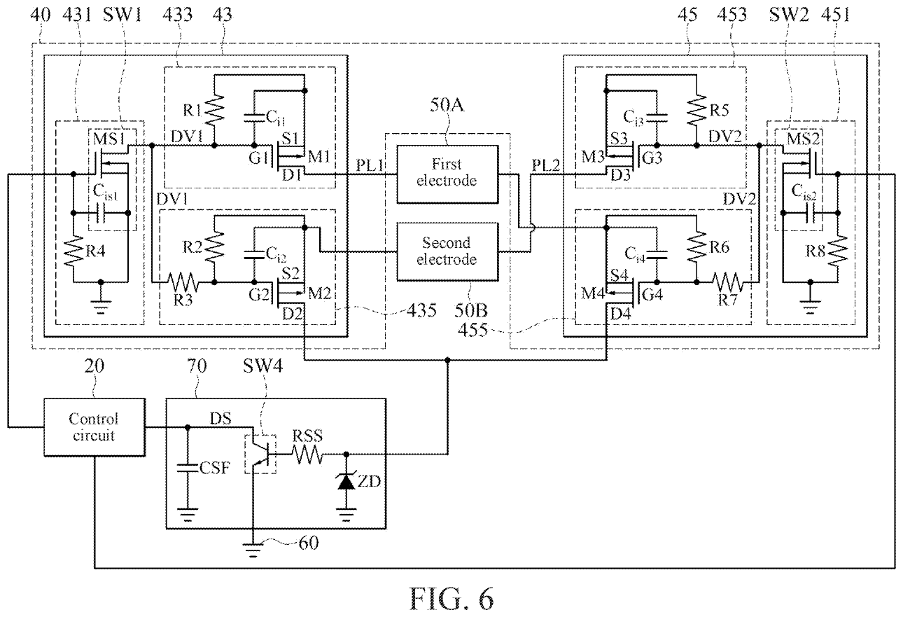

FIG. 6 is a schematic diagram of an electrode detection circuit 70 and part of an electrical stimulation circuit 40 according to some embodiments of the disclosure. In some embodiments, the electrical stimulation device 10 further includes the electrode detection circuit 70, located between the electrical stimulation circuit 40 and the reference ground terminal 60. The electrode detection circuit 70 generates a detection signal DS according to the electrical stimulation signal ES. Specifically, when detecting that the electrical stimulation circuit 40 emits the electrical stimulation signal ES (such as the second pulse circuit 435 transmits the second power supply signal HV from the second electrode 50B to the electrode detection circuit 70 and the fourth pulse circuit 455 transmits the second power supply signal HV from the first electrode 50A to the electrode detection circuit 70), the electrode detection circuit 70 generates the detection signal DS, and outputs the detection signal DS to the control circuit 20. The control circuit 20 determines, in response to the detection signal DS, that the first electrode 50A and the second electrode 50B are currently arranged in the arrangement region, and maintains operating of the electrical stimulation circuit 40. The control circuit 20 determines, when not receiving the detection signal DS, that the first electrode 50A and the second electrode 50B are currently not arranged in the arrangement region, and turns off the electrical stimulation circuit 40, to save energy and prevent the user from being electrically shocked.

As shown in FIG. 6, in some embodiments, the electrode detection circuit 70 includes a fourth changeover switch SW4 and a zener diode ZD. The fourth changeover switch SW4 is coupled to the electrical stimulation circuit 40, the zener diode ZD, the control circuit 20, and the reference ground terminal 60. The zener diode ZD turns on the fourth changeover switch SW4 according to the electrical stimulation signal ES, to enable the electrode detection circuit 70 to output the detection signal DS (such as a low-level logic signal) to the control circuit 20. The zener diode ZD turns off the fourth changeover switch SW4, when not receiving the electrical stimulation signal ES (such as not receiving the second power supply signal HV from the second pulse circuit 435 or the second power supply signal HV from the fourth pulse circuit 455), to disable the electrode detection circuit 70 from outputting the detection signal DS to the control circuit 20. In some embodiments, the fourth changeover switch SW4 may be implemented by an electronic switch, such as a transistor. In some embodiments, the electrode detection circuit 70 further includes a resistor (such as an eleventh resistor RSS), located between the fourth changeover switch SW4 and the zener diode ZD and configured to limit a ceiling of a current to prevent the electrode detection circuit 70 from being damaged due to an overload. In some embodiments, the electrode detection circuit 70 further includes a speed-up capacitor CSF, connected in parallel to the fourth changeover switch SW4, to increase the switching speed of the fourth changeover switch SW4.

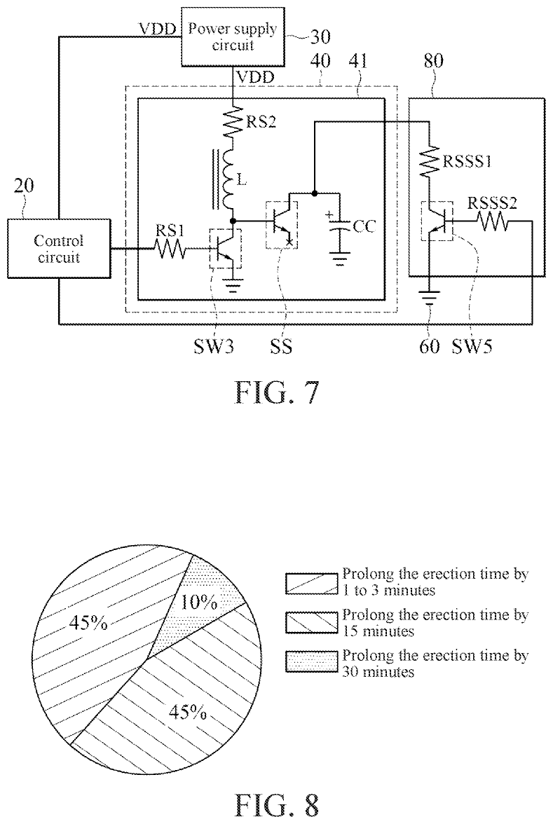

FIG. 7 is a schematic diagram of a discharging circuit 80 and part of an electrical stimulation circuit 40 according to some embodiments of the disclosure. In some embodiments, the electrical stimulation device 10 further includes a discharging circuit 80. When the control circuit 20 turns off the electrical stimulation circuit 40 (such as the control circuit 20 determines that the first electrode 50A and the second electrode 50B are currently not arranged in the arrangement region), the control circuit 20 controls the discharging circuit 80 to discharge the boost circuit 41, to save energy and prevent the user from being electrically shocked. In some embodiments, the discharging circuit 80 includes a fifth changeover switch SW5, coupled to the charging capacitor CC of the boost circuit 41, the reference ground terminal 60, and the control circuit 20. When the control circuit 20 turns off the electrical stimulation circuit 40, the control circuit 20 turns on the fifth changeover switch SW5, to discharge the charging capacitor CC to the reference ground terminal 60. In some embodiments, the discharging circuit 80 further includes two resistors (such as a twelfth resistor RSSS1 and a thirteenth resistor RSSS2), located between the fifth changeover switch SW5 and the control circuit 20 and located between the charging capacitor CC of the boost circuit 41 and the fifth changeover switch SW5 respectively and configured to limit a ceiling of a current to prevent the discharging circuit 80 from being damaged due to an overload. In some embodiments, the fifth changeover switch SW5 may be implemented by an electronic switch, such as a transistor.

In some embodiments, the electrical stimulation device 10 further includes a power switch (not shown in the figure). In response to a case that the power switch is in an on state, the control circuit 20 maintains operating of various circuits in the electrical stimulation device 10 (such as maintains operating of the electrical stimulation circuit 40). In response to a case that the power switch is in an off state, the control circuit 20 turns off various circuits in the electrical stimulation device 10 (such as turns off the electrical stimulation circuit 40 and discharges the boost circuit 41). In some embodiments, the power switch may be implemented by a push button switch. In some embodiments, the electrical stimulation device 10 further includes an indicating circuit (not shown in the figure), for generating different special effects in collaboration with different states of the power switch. In some embodiments, when the power switch is in the on state, the indicating circuit generates flashing light; and when the power switch is in the off state, the indicating circuit generates non-flashing light.

In some embodiments, the power supply circuit 30 includes a spare capacitor, for storing electric power to serve as spare electric power. A capacitance value of the spare capacitor is in a positive correlation with the first pulse frequency of the first pulse PL1 and the second pulse frequency of the second pulse PL2. For example, when the first pulse frequency of the first pulse PL1 (and/or the second pulse frequency of the second pulse PL2) is 200 thousand Hz, the capacitance value of the spare capacitor of the power supply circuit 30 may be 22 microfarads; when the first pulse frequency of the first pulse PL1 (and/or the second pulse frequency of the second pulse PL2) is 500 thousand Hz, the capacitance value of the spare capacitor of the power supply circuit 30 may be 47 microfarads; and when the first pulse frequency of the first pulse PL1 (and/or the second pulse frequency of the second pulse PL2) is 1 million Hz, the capacitance value of the spare capacitor of the power supply circuit 30 may be 100 microfarads.

It is worth being noted that, the transistor described in this specification may be implemented by an N-type transistor or a P-type transistor. When the transistor is implemented in a manner different from those described in the foregoing embodiments, how to properly adjust the architecture of the electrical stimulation device 10 can be deduced according to the disclosure.

Referring to FIG. 1B, FIG. 2, and FIG. 8 together, FIG. 8 is a schematic diagram of physiological symptom improvements of users after using an electrical stimulation device 10 implemented by a transcutaneous electrical stimulation device 10a according to some embodiments of the disclosure. It can be seen from FIG. 8 that, by adjusting various signal parameters (for example, a burst duty cycle, a burst frequency, a pulse duty cycle, a pulse frequency, and the like) of the electrical stimulation signal ES to values shown in the embodiments of this application (for example, adjusting the burst duty cycle of the first burst signal BS1 and/or the second burst signal BS2 to a range from 0.0005% to 50%, adjusting the burst frequency of the first burst signal BS1 and/or the second burst signal BS2 to a range from 0.1 Hz to 200 Hz, adjusting the pulse duty cycle of the first pulse PL1 and/or the second pulse PL2 to a range from 0.01% to 50%, adjusting the pulse frequency of the first pulse PL1 and/or the second pulse PL2 to a range from 1000 Hz to 10 million Hz, adjusting the burst width of the first burst signal BS1 and/or the second burst signal BS2 to a range from 25×10⁻⁹ seconds to 5 seconds, and/or adjusting the burst duty cycle of the first pulse PL1 and/or the second pulse PL2 to a range from 0.0005% to 50%), and by using a biphasic charge balance or a biphasic charge balance with delay, a to-be-stimulated target region of an organism can be better stimulated, to better alleviate physiological symptoms of the organism. Specifically, FIG. 8 shows statistical data about relieving premature ejaculation that is collected after 20 users use the electrical stimulation device 10 implemented by the transcutaneous electrical stimulation device 10a (as shown in FIG. 1B). After a user has performed an electrical stimulation for a short time (for example, 0.5 to 5 minutes) between the middle of a human penis (namely, reproductive organ P) and the corona of glans penis by using an electrical stimulation signal ES of the electrical stimulation device 10 implemented by the transcutaneous electrical stimulation device 10a, the dorsal penile nerve can undergo deactivation or desensitization, thereby alleviating an impulse of ejaculation. Compared with a case that the device is not used, it is found that, the erection time (the time starting from sexual intercourse to ejaculation, or from entry of the reproductive organ P into the vagina to ejaculation) of the penis of the user is prolonged. It can be seen from FIG. 8 that among the 20 users, the erection time of 45% users is prolonged by 1 to 3 minutes (represented by an oblique line from lower left to upper right), the erection time of 45% users is prolonged by 15 minutes (represented by an oblique line from upper left to lower right), and the erection time of 10% users is prolonged by 30 minutes (represented by dots).

In summary, according to some embodiments, the power supply circuit with a low power (such as the power ranges from 0.1 milliwatts to 30 milliwatts or ranges from 0.1 milliwatts to 30 watts) of the disclosure can meet electric power required for the electrical stimulation device for generating an high-frequency electrical stimulation signal (such as the pulse has a frequency ranging from 1000 Hz to 10 million Hz). In some embodiments, by adjusting the burst duty cycle of the first burst signal and/or the second burst signal to a range from 0.0005% to 50%, adjusting the burst frequency of the first burst signal and/or the second burst signal to a range from 0.1 Hz to 200 Hz, and adjusting the pulse duty cycle of the first pulse and/or the second pulse to a range from 0.01% to 50%, power consumption of the electrical stimulation device can be reduced and power can be saved. In some embodiments, when the first burst signal and the second burst signal are monophasic burst signals with opposite polarities, the first burst signal and the second burst signal that occur alternately can implement a biphasic charge balance, and the first burst signal and the second burst signal that occur alternately and are spaced apart by a period of time can implement a biphasic charge balance with delay. In this embodiment, damage caused by the electrical stimulation signal to a tissue of an organism can be avoided as much as possible. In some embodiments, when the first burst signal and the second burst signal are biphasic burst signals, the first pulses and the second pulses occurring alternately in each of the first burst signal and the second burst signal can implement a biphasic charge balance, and the first burst signal and the second burst signal that occur alternately and are spaced apart by a period of time can implement a biphasic charge balance with delay. In this way, damage caused by the electrical stimulation signal to a tissue of an organism can be avoided as much as possible.

What is claimed is:

1. An electrical stimulation device, comprising:
a control circuit;
a power supply circuit, providing a first power supply signal; and
an electrical stimulation circuit, controlled by the control circuit to generate an electrical stimulation signal according to the first power supply signal, wherein the electrical stimulation signal comprises a first burst signal and a second burst signal; a burst duty cycle of the first burst signal ranges from 0.0005% to 50%, and a burst frequency of the first burst signal ranges from 0.1 Hz to 200 Hz; the first burst signal is a first monophasic burst signal, the first monophasic burst signal comprises a plurality of first pulses, the second burst signal is a second monophasic burst signal, and the second monophasic burst signal comprises a plurality of second pulses; and the first pulses have a polarity opposite to that of the second pulses, the first pulses have a first pulse frequency, the second pulses have a second pulse frequency, the first pulse frequency and the second pulse frequency both range from 1000 Hz to 10 million Hz, and the first pulses have a pulse duty cycle ranging from 0.01% to 50%.

2. The electrical stimulation device according to claim 1, wherein the first burst signal and the second burst signal occur alternately and are spaced apart by a period of time, and the period of time ranges from 0 seconds to 5 seconds.

3. The electrical stimulation device according to claim 1, wherein when charges accumulated by the first monophasic burst signal or the second monophasic burst signal are greater than 20 μC, an electrical stimulation performed by the electrical stimulation signal is an electrical stimulation with sensation.

4. The electrical stimulation device according to claim 1, wherein the first burst signal has a burst width ranging from 25×10⁻⁹ seconds to 5 seconds, and each of the first pulses has a pulse width ranging from 25×10⁻¹⁶ seconds to 5×10⁻³ seconds.

5. The electrical stimulation device according to claim 1, wherein the first power supply signal has a power ranging from 0.1 milliwatts to 30 milliwatts.

6. The electrical stimulation device according to claim 1, wherein the first power supply signal has a power ranging from 0.1 milliwatts to 30 watts.

7. The electrical stimulation device according to claim 1, wherein the electrical stimulation circuit comprises:
a boost circuit, controlled by the control circuit to boost the first power supply signal to generate a second power supply signal, wherein the second power supply signal has a voltage ranging from 0.1 volts to 200 volts.

8. The electrical stimulation device according to claim 7, wherein the voltage ranging of the second power supply signal is from 0.1 volts to 100 volts.

9. The electrical stimulation device according to claim 7, further comprising: a first electrode and a second electrode with opposite polarities, wherein the electrical stimulation circuit further comprises:
a first output circuit, controlled by the control circuit to generate the first pulses at the first electrode and the second electrode according to the second power supply signal; and
a second output circuit, controlled by the control circuit to generate the second pulses at the first electrode and the second electrode according to the second power supply signal.

10. The electrical stimulation device according to claim 9, wherein the first output circuit comprises:
a first driving circuit, controlled by the control circuit to emit a first driving signal;
a first pulse circuit, receiving the second power supply signal according to the first driving signal and transmitting the second power supply signal to the first electrode; and
a second pulse circuit, transmitting the second power supply signal from the second electrode to a reference ground terminal according to the first driving signal, wherein
the control circuit controls, according to the first pulse frequency and the pulse duty cycle of the first pulses, the first driving circuit to emit the first driving signal, to enable the first output circuit to generate the first pulses at the first electrode and the second electrode.

11. The electrical stimulation device according to claim 10, wherein the first pulse circuit comprises:
a first transistor having a first input capacitor, wherein the first transistor comprises a first control terminal, a first input terminal, and a first output terminal, the first input capacitor is between the first control terminal and the first input terminal, the first control terminal receives the first driving signal, the first input terminal receives the second power supply signal, and the first output terminal transmits the second power supply signal to the first electrode; and
a first resistor, connected in parallel to the first input capacitor, wherein a product of a first resistance value of the first resistor and a first capacitance value of the first input capacitor is less than half of a pulse width of each of the first pulses.

12. The electrical stimulation device according to claim 10, wherein the second pulse circuit comprises:
a second transistor having a second input capacitor, wherein the second transistor comprises a second control terminal, a second input terminal, and a second output terminal, the second input capacitor is between the second control terminal and the second input terminal, the second control terminal receives the first driving signal, the second input terminal receives the second power supply signal from the second electrode, and the second output terminal transmits the second power supply signal to the reference ground terminal;
a second resistor, connected in parallel to the second input capacitor; and
a third resistor, located between the first driving circuit and the second control terminal, wherein a product of a third resistance value of the third resistor and a second capacitance value of the second input capacitor is less than half of a pulse width of each of the first pulses.

13. The electrical stimulation device according to claim 9, wherein the second output circuit comprises:
a second driving circuit, controlled by the control circuit to emit a second driving signal;
a third pulse circuit, receiving the second power supply signal according to the second driving signal and transmitting the second power supply signal to the second electrode; and
a fourth pulse circuit, transmitting the second power supply signal from the first electrode to a reference ground terminal according to the second driving signal, wherein
the control circuit controls, according to the second pulse frequency and the pulse duty cycle of the second pulses, the second driving circuit to emit the second driving signal, to enable the second output circuit to generate the second pulses at the first electrode and the second electrode.

14. The electrical stimulation device according to claim 13, wherein the third pulse circuit comprises:
a third transistor having a third input capacitor, wherein the third transistor comprises a third control terminal, a third input terminal, and a third output terminal, the third input capacitor is between the third control terminal and the third input terminal, the third control terminal receives the second driving signal, the third input terminal receives the second power supply signal, and the third output terminal transmits the second power supply signal to the second electrode; and
a fifth resistor, connected in parallel to the third input capacitor, wherein a product of a fifth resistance value of the fifth resistor and a third capacitance value of the third input capacitor is less than half of a pulse width of each of the second pulses.

15. The electrical stimulation device according to claim 13, wherein the fourth pulse circuit comprises:
a fourth transistor having a fourth input capacitor, wherein the fourth transistor comprises a fourth control terminal, a fourth input terminal, and a fourth output terminal, the fourth input capacitor is between the fourth control terminal and the fourth input terminal, the fourth control terminal receives the second driving signal, the fourth input terminal receives the second power supply signal from the first electrode, and the fourth output terminal transmits the second power supply signal to the reference ground terminal;
a sixth resistor, connected in parallel to the fourth input capacitor; and
a seventh resistor, located between the second driving circuit and the fourth control terminal, wherein a product of a seventh resistance value of the seventh resistor and a fourth capacitance value of the fourth input capacitor is less than half of a pulse width of each of the second pulses.

16. The electrical stimulation device according to claim 7, wherein the boost circuit comprises:
a third changeover switch, wherein the control circuit controls a changeover state of the third changeover switch;
an inductor, performing energy storage or release according to the changeover state and the first power supply signal, and generating a first charging signal to serve as the second power supply signal when the changeover state is off, wherein an inductance value of the inductor is in a positive correlation with the first pulse frequency and the second pulse frequency, and an impedance value of the inductor is in a negative correlation with the first pulse frequency and the second pulse frequency;
a charging capacitor, performing charging according to the first charging signal, and generating a second charging signal to serve as the second power supply signal when the changeover state is on, wherein a charging capacitance value of the charging capacitor is in a positive correlation with the first pulse frequency and the second pulse frequency; and
a blocking switch, located between a node between the third changeover switch and the inductor and the charging capacitor.

17. The electrical stimulation device according to claim 7, further comprising: an electrode detection circuit, located between the electrical stimulation circuit and a reference ground terminal and generating a detection signal according to the electrical stimulation signal, wherein the control circuit maintains operating of the electrical stimulation circuit in response to the detection signal and turns off the electrical stimulation circuit when the detection signal is not received.

18. An electrical stimulation device, comprising:
a control circuit;
a power supply circuit, providing a first power supply signal; and
an electrical stimulation circuit, controlled by the control circuit to generate an electrical stimulation signal according to the first power supply signal, wherein the electrical stimulation signal comprises a first burst signal and a second burst signal; a burst duty cycle of the first burst signal ranges from 0.0005% to 50%, and a burst frequency of the first burst signal ranges from 0.1 Hz to 200 Hz; each of the first burst signal and the second burst signal is a biphasic burst signal, and the biphasic burst signal comprises a plurality of first pulses and a plurality of second pulses occurring alternately; and the first pulses have a polarity opposite to that of the second pulses, the first pulses have a first pulse frequency, the second pulses have a second pulse frequency, the first pulse frequency and the second pulse frequency both range from 1000 Hz to 10 million Hz, and the first pulses have a duty cycle ranging from 0.01% to 50%.

19. The electrical stimulation device according to claim 18, wherein the first burst signal and the second burst signal occur alternately and are spaced apart by a period of time, and the period of time ranges from 0 seconds to 5 seconds.

20. The electrical stimulation device according to claim 18, wherein the first power supply signal has a power ranging from 0.1 milliwatts to 30 watts.

21. The electrical stimulation device according to claim 18, wherein the electrical stimulation circuit comprises:
a boost circuit, controlled by the control circuit to boost the first power supply signal to generate a second power supply signal, wherein the second power supply signal has a voltage ranging from 0.1 volts to 200 volts.
